(19) United States
(12) Patent Application Publication
(10) Pub. No.: US 2004/0115686 A1
(43) Pub. Date: Jun. 17, 2004

(54) Materials and methods to detect alternative splicing of mrna

(76) Inventors: Douglas Dolginow, Gaithersburg, MD (US); Lawrence Mertz, Gaithersburg, MD (US)

Correspondence Address: MORGAN LEWIS & BOCKIUS LLP, 1111 PENNSYLVANIA AVENUE NW, WASHINGTON, DC, 20004, US

(21) Appl. No.: 10/471,731

(22) PCT Filed: May 17, 2002
(86) PCT No.: PCT/US02/15649

(51) Int. Cl. C12Q 1/68; C12M 1/34

(52) U.S. Cl. 435/6; 435/287.2

(57) ABSTRACT

The invention relates generally to materials and methods for the detection and analysis of alternative splice variants of mRNA. In some embodiments, the present invention provides solid supports to which are affixed oligonucleotides having sequences complementary to predicted splice junction sequences. A splice variant profile may be prepared for a sample and compared to a corresponding profile of a normal and/or a disease tissue sample.

### PRIORITY APPLICATIONS

This application claims priority to U.S. Provisional Application No. 60/291,598, filed May 17, 2002, which is hereby incorporated by reference in its entirety.

### FIELD OF THE INVENTION

The invention relates generally to the field of molecular biology and gene expression. The invention includes to materials and methods to detect alternative splice variants of mRNA.

### BACKGROUND OF THE INVENTION

The majority of genes in eukaryotic organisms are discontinuous; wherein the primary nucleic acid sequence in the genome contains one or more sequences that are not reflected in the encoded polypeptide. When the genomic DNA is transcribed into RNA, the resultant RNA molecule contains sequences containing coding information (exons) and intervening, non-coding sequences (introns). After transcription into RNA, the introns are removed by splicing to generate mature messenger RNA (mRNA).

The emergence of human gene expression data and the map of the human genome are providing evidence that multiple mRNA transcripts are commonly expressed from a single human gene. The division of human genes into exon units, interrupted by introns, allows for the selective omission or inclusion of exons by a process commonly known as alternative splicing which creates related transcripts known as splice variants. The set of related mRNAs derived from a given gene by alternative splicing is called the transcriptome. As used herein, “splice variant profile” means the set of mRNAs expressed along with their expression levels.

Alternative splice variants have been associated with various disease states. For example, alternate splicing of the T-cell receptor zeta chain mRNA has been associated with lupus erythmatosis (Nanbiar, et al., Arthritis Rheum 44(6): 1336-1350, 2001), alternate splice variants of the vascular endothelial growth factor have been associated with osteoarthritis (Pufe, et al., Arthritis Rheum 44(5): 1082-1088, 2001), alternate splice variants of the presenilin-2 gene have been associated with some types of Alzheimer's disease (Sato, et al., J. Neurochem. 72(6):2498-2505, 1999), and alternate splice variants of CD44 have been associated with tumor progression (Gilcrease, et al., Cancer Research 86(11):2320-2326, 1999).

Numerous computational methods exist for predicting the location of possible splice sites in a transcribed RNA. For example, Thandaraj, et al., (Brief Bioinformatics, 1(4):343-56, Prediction of exact boundaries of exons, 2000), Kan, et al., (Genome Research 11(5):889-900, Gene structure prediction and alternative splicing analysis using genomically aligned ESTs, 2001), Salzberg, et al., (J. Computational Biology, 5(4):667-80, A decision tree system for finding genes in DNA, 1998), and Rampone, (Bioinformatics 14(8):676-84, Recognition of splice junctions on DNA sequences by BRAIN learning algorithm, 1998) all discuss various methods of predicting the actual site of a splice junction. These methods have varying degrees of success in predicting alternative splicing patterns; however, no currently available prediction algorithm is 100% accurate.

The inaccuracy of currently available computational methods has led some researchers to apply microarray technology in an effort to empirically determine the exons present in spliced mRNA. Shoemaker, et al., (Nature, 409:922-927, Experimental annotation of the human genome using microarray technology, 2001) describe the use of exon and tiling arrays to analyze and define full length transcripts on the basis of co-regulated expression of exons. Exon arrays were created using oligonucleotides having sequences derived from predicted exons. Tiling arrays were created using 60-mer oligonucleotides overlapped by ten bases across a 113.8 kb region of chromosome 22 including reverse complements of each tiling probe. Hu, et al. (Genome Research, 11(7):1237-1245, Predicting splice variant from DNA chip expression data, 2001) describe the analysis of rat gene expression patterns using a custom DNA chip having twenty pairs of probes-each pair consisting of a perfect match and mismatch probe-directed at the 3′-region of target mRNAs.

The microarrays used in the prior art have contained probes selected based on the predicted or known exon sequence or by using the entire genome sequence and overlapping the sequences of the probes. While either of these methods will permit the detection of an exon expressed in a mRNA sample, it provides no information concerning the arrangement of multiple exons that may be present in any given mRNA molecule. Thus, there exists a need in the art for improved microarrays and methods for detecting the presence of specific splice junctions and exons in mRNA.

### SUMMARY OF THE INVENTION

The present invention includes, in part, materials and methods for detecting alternatively spliced mRNA. In some embodiments, the present invention provides a solid support comprising a plurality of oligonucleotides, wherein each oligonucleotide has a sequence that specifically hybridizes to a splice junction sequence in a target mRNA. In some embodiments, the plurality of oligonucleotides may comprise at least

$${\sum }_{x = 1}^{n - 1} ( n - x ) + n$$

oligonucleotides, wherein n=the number of exons in the gene of interest. The solid supports of the invention may also comprise one or more oligonucleotides that specifically hybridize to an exon of the gene of interest.

In some embodiments of the present invention, the invention includes a solid support comprising at least two oligonucleotides, wherein a first oligonucleotide specifically hybridizes to a splice junction in a first mRNA transcribed from a first gene of interest and a second oligonucleotide that specifically hybridizes to a splice junction in a second mRNA transcribed from a second gene of interest. The genes may be the same or different. In some embodiments, the different genes may originate on different chromosomes. In some embodiments, the genes may be the result of a translocation event. In some embodiments, the first mRNA and the second mRNA have at least one exon in common. The solid supports according to the invention may further comprise a third and a fourth oligonucleotide, wherein the third oligonucleotide specifically hybridizes to an exon of the first gene and the fourth oligonucleotide specifically hybridizes to an exon of the second gene.

In another aspect of the invention, the present invention provides a solid support comprising oligonucleotides, wherein the oligonucleotides comprise at least one oligonucleotide that specifically hybridizes to each possible splice junction in a mRNA transcribed from a first gene of interest. The solid supports may optionally comprise additional oligonucleotides, preferably the additional oligonucleotides comprise at least one oligonucleotide that specifically hybridizes to each possible splice junction in a mRNA transcribed from a second gene of interest.

In another aspect of the present invention, the invention includes a method of detecting alternative spliced mRNA by contacting a solid support of the invention with a solution comprising nucleic acids representative of mRNA in a cell and detecting an alternatively spliced mRNA. The nucleic acids may be ribonucleic acids and/or deoxyribonucleic acids.

In another aspect of the present invention, the invention includes a method of detecting a pathological condition in a patient, wherein the pathological condition is characterized by alternative splice variants of one or more genes, by contacting a sample from the patient with a solid support according to the invention and detecting a level of expression of an alternative splice variant in the sample, wherein the expression level of the alternative splice variant is indicative of a pathological condition.

In another aspect of the invention, the invention provides a computer system that includes a database containing information identifying an expression level for one or more alternative splice variants of one or more mRNAs and a user interface to view the information. The computer system of the invention may optionally include a database that contains information identifying an expression level for an alternative splice variant in normal and/or disease tissue.

In another aspect, the present invention provides a method of identifying an agent that modulates a pathological condition by contacting a sample with the agent, determining a splice variant expression profile for at least one gene, comparing the splice variant profile to a splice variant profile obtained from a sample not treated with the agent, and determining a change in the splice variant profile, wherein a change in the splice variant profile is indicative of an agent that modulates the condition. The present invention also provides agents identified by this method. The agents may be optionally formulated for pharmaceutical use, for example, an effective amount of an agent to modulate a pathological condition may be combined with one or more pharmaceutically acceptable buffers, excipients, diluents and the like.

### DETAILED DESCRIPTION

I. General Description

The process of RNA splicing occurs in the nucleus and is directed by small nuclear riboproteins (snRNPs). These snRNPs are believed to recognize specific RNA sequences that are present at exon-intron boundaries that act as nucleation points to direct the splicing reaction. These conserved boundary sequences are known as 5′ splice (donor) and 3′ splice (acceptor) sites. In higher eukaryotes, the consensus sequences for splicing exon-intron sequences are as follows (the second line represents other possibilities at certain nucleotide positions):

5′.....CAG GUAAGU..................A........................UUUUUUUUUUUNUAG G.........3′
A G CCCCCCCCCCCC C A
\overline{5′EXON } \overline{ INTRON SEQUENCE } 3′EXON

Many different transcripts may result from variations in the snRNP-directed splicing of an RNA molecule transcribed from a gene unit that contains multiple exons. For example, if the genomic organization of a gene is as follows:

5′....EXON1.............EXON2..................EXON3.....EXON4....................3′

Alternative spliced transcripts containing one or more of the exons, i. e. transcripts containing exon 1, exons 1 and 2, exons 1, 2 and 3 etc., may be formed. The present invention provides materials and methods for the detection and analysis of these alternative spliced transcripts also referred to herein as splice variants.

Definitions

In the description that follows, numerous terms and phrases known to those skilled in the art are used. In the interest of clarity and consistency of interpretation, the definitions of certain terms and phrases as used herein are provided.

As used herein, oligonucleotide sequences that are complementary to one or more of the nucleic acids (DNA, mRNA, cDNA, rRNA etc.) described herein, such as sequence comprising a splice junction site, refers to oligonucleotides that are capable of hybridizing under stringent conditions to at least part of the nucleotide sequence of said nucleic acids. Such hybridizable oligonucleotides will typically exhibit at least about 75% sequence identity at the nucleotide level to said nucleic acids, preferably about 80% or 85% sequence identity or more preferably about 90% or 95% or more sequence identity.

As used herein, “bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g., probes directed to nucleic acids of the opposite sense or to genes not found in the sample such as bacterial genes where the sample is mammalian nucleic acids). Background can also be calculated as the average signal intensity produced by regions of the array that lack any probes at all.

As used herein, the terms “gene” and “gene unit” refer to a segment of DNA comprising both coding sequences (exons) and non-coding sequences (introns) that occupies a particular chromosomal locus and contains all the information for the coding of at least one mRNA product (unless intergenic exon splicing occurs). Said mRNA products may comprise differential arrangements of the exons of the gene, resulting in the encoding of differential polypeptide or protein products that are splice variants of one another. As used herein, the terms “gene” and “gene unit” also include the term “allele,” which, as used herein, encompasses naturally or artificially occurring alternative forms of a gene occupying a particular chromosomal locus.

The phrase “hybridizing specifically to” or “specifically hybridize” refers to the binding, duplexing or hybridizing of a molecule substantially to or only to a particular nucleotide sequence or sequences under stringent conditions when that sequence is present in a complex mixture (e.g., total cellular) DNA or RNA.

Assays and methods of the invention may utilize available formats to simultaneously screen at least about 100, preferably about 1000, more preferably about 10,000 and most preferably about 1,000,000 or more different nucleic acid hybridizations.

The terms “mismatch control” or “mismatch probe” refer to a probe whose sequence is deliberately selected not to be perfectly complementary to a particular target sequence. For each mismatch (MM) control in a high-density array there typically exists a corresponding perfect match (PM) probe that is perfectly complementary to the same particular target sequence. The mismatch may comprise one or more bases.

While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe and the like. A perfect match control or perfect match probe is, however, distinguished from a “mismatch control” or “mismatch probe.”

As used herein, the term “predicted” refers to any nucleic acid sequence being investigated, studied, probed or tested for being adjacent to the splice junction site of two exons at either the 5′ or 3′ side.

As used herein a “probe” is defined as a nucleic acid, preferably an oligonucleotide, capable of binding to a target nucleic acid of complementary sequence through one or more types of chemical bonds, usually through complementary base pairing, usually through hydrogen bond formation. As used herein, a probe may include natural (i.e., A, G, U, C or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

As used herein, the term “splice variants” within a gene or allele refers to related transcripts which are products of alternative splicing between exons of said gene or allele resulting in the selective omission or inclusion of exons. Splice variants also include the products of the fusion of exons from at least two different genes or alleles. Said different genes may originate on the same chromosome and be adjacent or in close proximity to one another. Said different genes may alternatively originate on the same or different chromosomes and their exons may be brought into proximity with one another by, for example, at least one of a translocation, crossover, chiasma, deletion, insertion, substitution, inversion, intrachromosomal rearrangement, intrachange, or recombination event.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tₘ) for the specific sequence at a defined ionic strength and pH.

Typically, stringent conditions will be those in which the salt concentration is at least about 0.01 to 1.0 M sodium ion concentration (or other salts) at pH 7.0 to 8.3 and the temperature is at least about 30° C. for short probes (e.g., 10 to 50 nucleotide). Stringent conditions may also be achieved with the addition of destabilizing agents such as formamide.

Some preferred examples of “stringent conditions” include those that (1) employ low ionic strength and high temperature for washing, for example, 0.015 M NaCl/0.0015 M sodium citrate/0.1% SDS at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is hybridization in 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS. A skilled artisan can readily determine and vary the stringency conditions appropriately to obtain a clear and detectable hybridization signal.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical subunit (e.g., nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

Homology or identity may be determined by BLAST (Basic Local Alignment Search Tool) analysis using the algorithm employed by the programs blastp, blastn, blastx, tblastn and tblastx (Karlin et al., (1990) Proc. Natl. Acad. Sci. USA 87, 2264-2268 and Altschul, (1993) J. Mol. Evol. 36, 290-300, fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., ((1994) Nature Genet. 6, 119-129) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) Proc. Natl. Acad. Sci. USA 89, 10915-10919, fully incorporated by reference). Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

II. Specific Embodiments

Currently, commonly used array technologies that detect expression of mRNAs are not optimized to detect expression of alternatively spliced transcripts. Many arrays usually either array specific short DNA sequences (oligos) that are complementary to a portion of an exon in a gene, or they utilize larger cDNAs, to which many differently spliced transcripts can hybridize, thus making any conclusions concerning splice variants very tenuous.

One embodiment of the present invention is a microarray and method that can more accurately detect and quantify the expression of alternatively spliced mRNAs by arraying both sequences specific to and preferably within each exon, as well as DNA sequences that are specific to the predicted exon-exon junctions or splice junctions. For example, if a genomic unit is described as having exons A, B and C, DNA sequences specific to one or more of the exons may be arrayed and, in addition, sequences pertaining to one or more of the exon junctions AB, BC and AC are also arrayed. This approach may be expanded for more complex genes containing more exons as shown in Table 1.

TABLE 1

| | | | Total |
|---|---|---|---|
| Number of | Exon sequences | Exon junction sequences | sequences |
| Exons | arrayed | arrayed | arrayed |
| 3 | A, B, and C | AB, AC, and BC | 6 |
| 4 | A, B, C, and D | AB, AC, AD, BC, BD, and | 10 |
| | | CD | |
| 5 | A, B, C, D, and E | AB, AC, AD, AE, BC, BD, | 15 |
| | | BE, CD, CE, and DE | |
| 6 | A, B, C, D, E, | AB, AC, AD, AE, AF, BC, | 21 |
| | and F | BD, BE, BF, CD, CE, CF, | |
| | | DE, DF, and EF | |

Those skilled in the art will appreciate that this approach may be expanded to as many exons as are potentially present in a target gene. The minimum number of sequences required in order to have one sequence specific to each possible exon-exon combination which may then be used to detect the presence or absence of one or more junctions can be determined using the following formula:

$${\sum }_{x = 1}^{n - 1} ( n - x ) + n$$

where n=the number of exons in the gene of interest

In another embodiment of the present invention, exon shuffling may be detected using sequences designed to hybridize with one or more of the exon-exon junctions that result from the shuffled exons. For example, if a gene of interest contained exons A, B, C, and D, the sequences to be arrayed might include one or more sequences designed to detect one or more of the following exon-exon junctions: AB, AC, AD, BC, BD, CD, BA, CA, DA, CB, DB, and DC (a total of 12).

In the case of exon shuffling, the minimum number of sequences required in order to have one sequence specific to each possible exon-exon combination which may then be used to detect the presence or absence of one or more junctions can be determined using the following formula:

$$2 [ {\sum }_{x = 1}^{n - 1} ( n - x ) ] + n$$

where n=the number of exons in the gene

In another embodiment of the present invention, a combinatorial approach may be used to detect all possible exon-exon junctions resulting from alternative splicing and/or crossover events involving more than one gene unit. This embodiment of the invention will be particularly useful to detect transcriptomes resulting from gene shuffling or chromosomal cross over events in the human genome that are often involved in disease.

For example, in the case of two gene units the first having exons A, B, and C and the second having exons X, Y, and Z, the sequences to be arrayed might include one or more sequences designed to detect one or more of the following exon-exon junctions: AB, AC, BC, BA, CA, CB (a total of 6 specific for the possible junctions of the first gene), XY, XZ, YZ, YX, ZX, and ZY (a total of 6 specific for the possible junctions of the second gene) and AX, AY, AZ, BX, BY, BZ, CX, CY, CZ, XA, YA, ZA, XB, YB, YC, ZA, ZB, and ZC (a total of 18 for possible junctions involving both genes).

The following formula can be used to predict the minimum number of oligonucleotide sequences that must be arrayed in order to detect all possible exon-exon junctions involving two genes:

$$2 [ {\sum }_{X = 1}^{N - 1} ( N - X ) ] + N + 2 [ {\sum }_{X = 1}^{P - 1} ( P - X ) ] + P + [ N \cdot 2 ( P ) ]$$

where N=number of exons in gene ABC

where P=number of exons in gene XYZ.

In one embodiment of the invention, the splice variant is detected using at least one oligonucleotide species comprising at least all or a fraction of the exon predicted to be 5′ of the splice site and at least about a fraction of or all of the exon predicted to be 3′ of the splice site. In some embodiments, the oligonucleotides include at least part of the exons that are 5′ to the exon that is predicted to be immediately 5′ of the splice of interest. In some embodiments, the oligonucleotides include at least part of the exons that are 3′ to the exon that is predicted to be immediately 3′ of the splice of interest. In particular embodiments, said oligonucleotide comprises about the 3′ ½, ¼, or 1/10 of the exon predicted to be 5′ of the splice. In particular embodiments, said oligonucleotide comprises about the 5′ ½, ¼, or 1/10 of the exon predicted to be 3′ of the splice.

In a particular embodiment, said oligonucleotide comprises at least about the 3′-terminal 50 nucleotides of the exon predicted to be 5′ of the splice. In another particular embodiment, said oligonucleotide comprises at least about the 3′-terminal 30 nucleotides of the exon predicted to be 5′ of the splice. In still another particular embodiment, said oligonucleotide comprises at least about the 3′-terminal 25 nucleotides of the exon predicted to be 5′ of the splice. In yet another particular embodiment, said oligonucleotide comprises at least about the 3′-terminal 20 nucleotides of the exon predicted to be 5′ of the splice. In even another particular embodiment, said oligonucleotide comprises at least about the 3′-terminal 15 nucleotides of the exon predicted to be 5′ of the splice. In a preferred embodiment, said oligonucleotide comprises at least about the 3′-terminal 12 nucleotides of the exon predicted to be 5′ of the splice. In another preferred embodiment, said oligonucleotide comprises at least about the 3′-terminal 10 nucleotides of the exon predicted to be 5′ of the splice. In still another preferred embodiment, said oligonucleotide comprises at least about the 3′-terminal 5 nucleotides of the exon predicted to be 5′ of the splice.

In a particular embodiment, said oligonucleotide comprises at least about the 5′-terminal 5 nucleotides of the exon predicted to be 3′ of the splice. In another particular embodiment, said oligonucleotide comprises at least about the 5′-terminal 10 nucleotides of the exon predicted to be 3′ of the splice. In still another particular embodiment, said oligonucleotide comprises at least about the 5′-terminal 12 nucleotides of the exon predicted to be 3′ of the splice. In yet another particular embodiment, said oligonucleotide comprises at least about the 5′-terminal 15 nucleotides of the exon predicted to be 3′ of the splice. In even another particular embodiment, said oligonucleotide comprises at least about the 5′-terminal 20 nucleotides of the exon predicted to be 3′ of the splice. In yet still another particular embodiment, said oligonucleotide comprises at least about the 5′-terminal 25 nucleotides of the exon predicted to be 3′ of the splice. In even still another particular embodiment, said oligonucleotide comprises at least about the 5′-terminal 30 nucleotides of the exon predicted to be 3′ of the splice. In another particular embodiment, said oligonucleotide comprises at least about the 5′-terminal 50 nucleotides of the exon predicted to be 3′ of the splice. In any of these embodiments, said oligonucleotide may further comprise a deletion of about the 3′-terminal 1, 2, 3, 4, 5, 6, 7, 8, 9, or 10 nucleotides of the exon predicted to be 5′ of the splice, with the proviso that at least 1 nucleotide (5′ to the deletion) of said 5′ exon remains, and/or a deletion of about the 5′-terminal 1, 2, 3, 4, 5, 6, 7, 8, 9, or 10 nucleotides of the exon predicted to be 3′ of the splice, with the proviso that at least 1 nucleotide (3′ to the deletion) of said 3′ exon remains.

In another embodiment of the invention, oligonucleotides having sequences that specifically hybridize to sequences surrounding the predicted exon-exon splice junction may be arrayed. In some embodiments of this type, oligonucleotides may be selected such that the sequence of each oligonucleotide overlaps—i. e., has sequence in common with—other nucleotides that are arrayed. This is referred to as tiling of the oligonucleotides (see, for example, U.S. Pat. No. 5,837,832). This embodiment may be useful in identifying exon-exon splice junctions that are difficult to predict accurately based upon currently available prediction algorithms.

Use of tiled oligonucleotides permits the creation of a clustered set that will help capture the splice regions. To create this set of clustered sequences, predictions of splice regions may be made from the genomic DNA using currently available splice junction prediction algorithms. After the predicted sites are identified, a clustered set of oligonucleotides spanning a region around the predicted site may be arrayed. Thus, for a given target sequence encompassing a predicted splice junction, a set of oligonucleotides of length L may be synthesized such that each contains a sequence complementary to a portion of the target sequence. The first oligonucleotide in the set may have a sequence complementary to the target sequence starting at starting at nucleotide X of the target sequence, while the next oligonucleotide in the series may have a sequence complementary to the target sequence starting at starting at nucleotide X+N of the target sequence. N can be any number from 1 to L and is preferably in the range of about 1 to about 15 and most preferably is in the range of about 1 to 5. In some embodiments, the region selected for the clustered set may be from about 1 kb 5′ of the predicted splice site to about 1 kb 3′ of the predicted splice site in the genomic DNA sequence of the gene. In one embodiment the cluster set may begin with an oligonucleotide comprising at least about 50 nucleotides of the 3′ end of the exon predicted to be 5′ of the splice site. In another embodiment, the cluster set may begin with an oligonucleotide comprising at least about 30 nucleotides of said 3′ end. In still another embodiment, the cluster set may begin with an oligonucleotide comprising at least about 25 nucleotides of said 3′ end. In yet another embodiment, the cluster set may begin with an oligonucleotide comprising at least about 20 nucleotides of said 3′ end. In even another embodiment, the cluster set may begin with an oligonucleotide comprising at least about 15 nucleotides of said 3′ end. In a preferred embodiment, the cluster set may begin with an oligonucleotide comprising at least about 12 nucleotides of said 3′ end. In another preferred embodiment, the cluster set may begin with an oligonucleotide comprising at least about 10 nucleotides of said 3′ end. In still another preferred embodiment, the cluster set may begin with an oligonucleotide comprising at least about 5 nucleotides of said 3′ end.

The cluster set may also include oligonucleotides that extend at least about 5 nucleotides into the 5′ end of the exon predicted to be 3′ of the splice site. In another embodiment, said oligonucleotides extend at least about 10 nucleotides into the 5′ end of the exon predicted to be 3′ of the splice site. In still another embodiment, said oligonucleotides extend at least about 12 nucleotides into the 5′ end of the exon predicted to be 3′ of the splice site. In yet another embodiment, said oligonucleotides extend at least about 15 nucleotides into the 5′ end of the exon predicted to be. 3′ of the splice site. In even another embodiment, said oligonucleotides extend at least about 20 nucleotides into the 5′ end of the exon predicted to be 3′ of the splice site. In another embodiment, said oligonucleotides extend at least about 25 nucleotides into the 5′ end of the exon predicted to be 3′ of the splice site. In another embodiment, said oligonucleotides extend at least about 30 nucleotides into the 5′ end of the exon predicted to be 3′ of the splice site. In another embodiment, said oligonucleotides extend at least about 50 nucleotides into the 5′ end of the exon predicted to be 3′ of the splice site.

Said oligonucleotides of said cluster set may all be of the same length or of different lengths, may begin with the same nucleotide of the exon 5′ of the splice or may begin with different nucleotides of said 5′ exon, and may end with the same nucleotide of the exon 3′ of the splice or may end with different nucleotides of said 3′ exon. In any of these embodiments, said oligonucleotide may further comprise a deletion of about the 3′-terminal 1, 2, 3, 4, 5, 6, 7, 8, 9, or 10 nucleotides of the exon predicted to be 5′ of the splice, with the proviso that at least 1 nucleotide (5′ to the deletion) of said 5′ exon remains, and/or a deletion of about the 5′-terminal 1, 2, 3, 4, 5, 6, 7, 8, 9, or 10 nucleotides of the exon predicted to be 3′ of the splice, with the proviso that at least 1 nucleotide (3′ to the deletion) of said 3′ exon remains.

In some embodiments, the microarrays of the present invention may incorporate one or more oligonucleotides having sequences that are not predicted to be complementary to a splice junction sequence. For example, one or more oligonucleotides might be arrayed that have sequences predicted to be complementary to a sequence in a particular exon. Oligonucleotides of this type may be designed so as to be complementary to a sequence that is entirely within the target exon, i. e., does not extend into any splice junction sequences. Alternatively, oligonucleotides of this type may contain sequences predicted to be complementary to all or a portion of the splice junction as well as to a portion or all of the exon.

In some embodiments, oligonucleotides may be arrayed that contain sequences predicted to be complementary to a sequence present in an intron. Oligonucleotides of this type may be designed to be complementary to a sequence entirely contained within the intron. Alternatively, oligonucleotides of this type may be complementary to all or a portion of the intron and to all or a portion of a predicted splice junction sequence.

In some embodiments, oligonucleotides may be arrayed that contain a sequence designed to be complementary to all or a portion of an exon, all or a portion of a splice junction and all or a portion of an intron. Such oligonucleotides may span a genomic sequence that includes a predicted splice site as well as all or a portion of the exon and the intron that surround the splice site.

In some embodiments, oligonucleotides specific for exon sequences, and in some cases oligonucleotides specific for intron sequences, may be arrayed along with oligonucleotides specific for splice junction sequences. Arrays of this type will provide detailed information concerning the composition of the various alternative spliced mRNAs that may be generated in a particular transcriptome.

In some embodiments, an array of the present invention may comprise oligonucleotides such as those described above-complementary to a splice junction, exon, intron and/or combinations thereof-that are designed to be complementary to an individual gene. A single array may contain oligonucleotides for a number of individual genes. In addition, arrays may be designed to detect shuffled exons as described above. Such arrays may include oligonucleotides designed to be complementary to exons, introns and/or splice junctions from two or more different genes.

Uses of Splice Variants

The present invention provides materials and methods to identify those genes that express multiple splice variants and to identify which of the theoretically possible splice variants are actually expressed in any given tissue. One of skill in the art can select one or more of the genes identified as having splice variants and use the information and methods provided herein to interrogate or test a particular sample. For a particular interrogation of two conditions or tissue sources, it is desirable to select those genes that display a difference in the presence and/or amount of splice variants produced between the two conditions or sources. These differences may be in the amount of a particular splice variant in one sample versus another or in the distribution of splice variants in one sample versus another.

Splice variants also include the products of the fusion of exons from at least two different genes. Said different genes may originate on the same chromosome and be adjacent or in close proximity to one another. Said different genes may alternatively originate on the same or different chromosomes and their exons may be brought into proximity with one another by, for example, at least one of a translocation, crossover, chiasma, deletion, insertion, substitution, inversion, intrachromosomal rearrangement, intrachange, or recombination event.

One example of the use of the materials and methods of the present invention to predict disease states is in the diagnosis of those diseases described in the background section. Other disease states include, but are not limited to, a number of carcinomas, sarcomas, leukemias, lymphomas, pancreatitis and polycystic kidney disease.

For instance, a tissue sample or other sample from a patient may be assayed by any of the methods described herein or otherwise known to those skilled in the art, and the presence and/or level of expression of one or more splice variants of one or more genes of interest may be compared to that of normal cells and/or cells derived from a disease tissue sample in order to determine whether a given sample contains disease tissue. Comparison of the may be done with the aid of a computer and databases as described herein.

Use of the Splice Variants for Monitoring Disease Progression

The presence of a particular splice variant and/or level of expression of one or more splice variants may also be used as markers for the monitoring of disease progression, for instance, the amount of the splice variant of CD44 associated with tumor progression may be determined. To monitor the progression, a tissue sample or other sample from a patient may be assayed by any of the methods known to those of skill in the art, and the presence and/or amount of one or more splice variants of one or more genes may be determined in the sample and may be compared to those found in normal tissue, tissue from a diseased individual or both. Comparison of the data may be done by researcher or diagnostician or may be done with the aid of a computer and databases as described herein.

Use of the Splice Variants for Screening of Agents that Modulate the Splice Variant Profile

Potential agents can be screened to determine if application of the agent alters the splice variant profile of one or more genes. This may be useful, for example, in determining whether a particular drug is effective in treating a particular patient with a disease, for example a tumor. In the case where the potential agent affects the splice variant profile such that the profile returns to normal or is altered to be more like normal, the agent is indicated in the treatment of the disease. Similarly, an agent that induces the expression of a splice variant profile that is similar to that expressed in a disease state may be contraindicated.

According to the present invention, a gene identified as having one or more alternative splice variants may be used as the basis of an assay to evaluate the effects of a candidate drug or agent on a cell, for example on a diseased cell. Alternatively, according to the present invention, a coding sequence which is the product of alternative splice variants of at least two different genes may be used as the basis of an assay to evaluate the effects of a candidate drug or agent on a cell, for example on a diseased cell. Said different genes includes genes which originate on the same chromosome or on different chromosomes. A candidate drug or agent can be screened for the ability to modulate the production of one or more alternatively spliced mRNA molecules or the proteins translated from them. According to the present invention, one can also compare the specificity of a drug's effects by looking at the number and/or level of splice variants affected by the drug and comparing them to the number of splice variants affected by a different drug. A more specific drug will affect fewer splice variants. Similar sets of splice variants affected by two drugs indicates a similarity of effects.

Assays to monitor the expression of one or more splice variants may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

Agents that are assayed in the above methods can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent□s action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates, lipids, oligonucleotides and covalent and non-covalent combinations thereof. Dominant negative proteins, DNA encoding these proteins, antibodies to these proteins, peptide fragments of these proteins or mimics of these proteins may be introduced into cells to affect fiction. “Mimic” as used herein refers to the modification of a region or several regions of a peptide molecule to provide a structure chemically different from the parent peptide but topographically and functionally similar to the parent peptide (see Grant, (1995) in Molecular Biology and Biotechnology Meyers (editor) VCH Publishers). A skilled artisan can readily recognize that there is no limit as to the structural nature of the agents of the present invention.

Uses for Agents that Modulate the Splice Variant Profile of a Transcriptome

As provided for herein, agents that up- or down-regulate or modulate the production of one or more splice variants thereby altering the splice variant profile, may be used to modulate biological and pathologic processes associated with one or more of the splice variants affected.

As used herein, a subject can be any mammal, so long as the mammal is in need of modulation of a pathological or biological process mediated by a protein of the invention. The term “mammal” is defined as an individual belonging to the class Mammalia. The invention is particularly useful in the treatment of human subjects.

Pathological processes refer to a category of biological processes that produce a deleterious effect. For example, expression of a particular splice variant may be associated with a disease or other pathological condition. As used herein, an agent is said to modulate a pathological process when the agent reduces the degree or severity of the process. For instance, tumor progression may be prevented or slowed by the administration of agents which up- or down-regulate or modulate in some way the production of splice variants of CD44.

The agents of the present invention can be provided alone, or in combination with other agents that modulate a particular pathological process. For example, an agent of the present invention can be administered in combination with other known drugs. As used herein, two agents are said to be administered in combination when the two agents are administered simultaneously or are administered independently in a fashion such that the agents will act at the same time.

The agents of the present invention can be administered via parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired. The present invention further provides compositions containing one or more agents that modulate the splice variant profile of one or more genes. While individual needs vary, determination of optimal ranges of effective amounts of each component is within the skill. of the art. Typical dosages comprise 0. 1 to 100 μg/kg body wt. The preferred dosages comprise 0.1 to 10 μg/kg body wt. The most preferred dosages comprise 0.1 to 1 μg/kg body wt.

In addition to the pharmacologically active agent, the compositions of the present invention may contain suitable pharmaceutically acceptable carriers comprising excipients and auxiliaries that facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances that increase the viscosity of the suspension including, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

In practicing the methods of this invention, the compounds of this invention may be used alone or in combination, or in combination with other therapeutic or diagnostic agents. In certain preferred embodiments, the compounds of this invention may be coadministered along with other compounds typically prescribed for these conditions according to generally accepted medical practice. The compounds of this invention can be utilized in vivo, ordinarily in mammals, such as humans, sheep, horses, cattle, pigs, dogs, cats, rats and mice, or in vitro.

Diagnostic Methods

Since alterations in the splice variant profiles of various genes have been associated with disease states, the materials and methods of the present invention may be used to diagnosis disease states and/or their progression. One means of diagnosing diseases using the materials and methods of the present invention involves obtaining disease tissue from living subjects. Such tissue samples may be obtained by any conventional means, for example, by biopsy. When possible, urine, blood or peripheral lymphocyte samples may be used as the tissue sample in the assay.

The use of molecular biological tools has become routine in forensic technology. For example, the materials and methods of the present invention may be used to determine the splice variant profile of one or more genes in forensic/pathology specimens. Further, nucleic acid assays may be carried out by any means of conducting a transcriptional profiling analysis. In addition to nucleic acid analysis, forensic methods of the invention may target the proteins of the invention, particularly proteins produced from an alternative splice variant.

Methods of the invention may involve treatment of tissues with collagenases or other proteases to make the tissue amenable to cell lysis (Semenov D E et al., (1987) Biull Eksp Biol Med 104:113-116). Further, it is possible to obtain biopsy samples from different regions of a target tissue for analysis.

Assays to detect nucleic acid or protein molecules of the invention may be in any available format. Typical assays for nucleic acid molecules include hybridization or PCR based formats. Typical assays for the detection of proteins, polypeptides or peptides of the invention include the use of antibody probes in any available format such as in situ binding assays, etc. See Harlow & Lane, (1988) Antibodies—A Laboratory Manual, Cold Spring Harbor Laboratory Press. In preferred embodiments, assays are carried-out with appropriate controls.

Assay Formats

The genes identified as undergoing alternative splicing may be used in a variety of nucleic acid detection assays to detect or quantify the expression level of one or more splice variants in a given sample. For example, traditional Northern blotting, nuclease protection, RT-PCR and differential display methods may be used for detecting splice variant expression levels.

The protein products of the alternative splice variants identified using the materials and methods of the present invention can also be assayed to determine the amount of expression. Methods for assaying for a protein include Western blot, immunoprecipitation, and radioimmunoassay. It is preferred, however, that the mRNA be assayed as an indication of expression. Methods for assaying for mRNA include northern blots, slot blots, dot blots, and hybridization to an ordered array of oligonucleotides. Any method for specifically and quantitatively measuring a specific protein or mRNA or DNA product can be used. However, methods and assays of the invention are most efficiently designed with array or chip hybridization-based methods for detecting the splice variant profile of a large number of genes.

Once an alternative splice variant has been identified and characterized using the materials and methods of the present invention, any hybridization assay format may be used to detect these variants in a sample of interest. Such formats include solution-based and solid support-based assay formats. A preferred solid support is a high-density array also known as a DNA chip or a gene chip. In one assay format, gene chips containing probes to at least one predicted splice junction may be used to directly monitor or detect changes in splice variant profile in a treated or exposed cell as described herein.

Additional assay formats may be used to monitor the ability of the agent to modulate the expression of a splice variant. For instance, as described above, mRNA expression may be monitored directly by hybridization of probes to the nucleic acids of the invention. Cell lines are exposed to an agent to be tested under appropriate conditions and time and total RNA or mRNA is isolated by standard procedures such those disclosed in Sambrook et al., (1989) Molecular Cloning—A Laboratory Manual, Cold Spring Harbor Laboratory Press). In some embodiments, it may be desirable to amplify one or more of the RNA molecules isolated prior to application of the RNA to the gene chip. Using techniques well known in the art, the RNA may be reverse transcribed and amplified in the form of DNA or may be reverse transcribed into DNA and the DNA used as a template for transcription to generate recombinant RNA. Any method that results in the production of a sufficient quantity of nucleic acid to be hybridized effectively to the gene chip may be used.

In another format, cell lines that contain reporter gene fusions between the alternative splice variants and, optionally their 3′ and/or 5′ regulatory regions and any assayable fusion partner may be prepared. Numerous assayable fusion partners are known and readily available including the firefly luciferase gene and the gene encoding chloramphenicol acetyltransferase (Alam et al., (1990) Anal. Biochem. 188, 245-254). Cell lines containing the reporter gene fusions are then exposed to the agent to be tested under appropriate conditions and time. Differential expression of the reporter gene between samples exposed to the agent and control samples identifies agents that modulate the expression of the nucleic acid.

In another assay format, cells or cell lines are first identified which express one or more of the splice variants of the invention physiologically. Cells and/or cell lines so identified would preferably comprise the necessary cellular machinery to ensure that the transcriptional and/or translational apparatus of the cells would faithfully mimic the response of normal or diseased tissue to an exogenous agent. Such machinery would likely include appropriate surface transduction mechanisms and/or cytosolic factors. The cells and/or cell lines may then be contacted with an agent and the expression of one or more of the splice variants of interest may then be assayed. The splice variants may be assayed at the mRNA level and/or at the protein level.

In some embodiments, such cells or cell lines may be transduced or transfected with an expression vehicle (e.g., a plasmid or viral vector) containing an expression construct comprising an operable 5′-promoter containing end of a gene having a splice variant of interest identified using the materials and methods of the invention fused to one or more nucleic acid sequences encoding one or more antigenic fragments. The construct may comprise all or a portion of the coding sequence of one or more exons of the splice variant of interest that may be positioned 5′- or 3′- to a sequence encoding an antigenic fragment. The coding sequence of one or more of the exons of the splice variant may be translated or un-translated after transcription of the gene fusion. At least one antigenic fragment may be translated. The antigenic fragments are selected so that the fragments are under the transcriptional control of the promoter of the splice variant of interest and are expressed in a fashion substantially similar to the expression pattern of the gene of interest. The antigenic fragments may be expressed as polypeptides whose molecular weight can be distinguished from the naturally occurring polypeptides. In some embodiments, gene products of the invention may further comprise an immunologically distinct tag. Such a process is well known in the art (see Sambrook et al., (1989) Molecular Cloning—A Laboratory Manual, Cold Spring Harbor Laboratory Press).

Cells or cell lines transduced or transfected as outlined above are then contacted with agents under appropriate conditions; for example, an agent may comprise a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and serum incubated at 37° C. The conditions may be modulated as deemed necessary by one of skill in the art. Subsequent to contacting the cells with the agent, the cells may be disrupted and the polypeptides of the lysate may be fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient is contacted with the cells and an increase or decrease in the immunologically generated signal from the “agent-contacted” sample compared to the control will be used to distinguish the effectiveness of the agent.

Another embodiment of the present invention provides methods for identifying agents that modulate the levels, concentration or at least one activity of a protein(s) encoded by a splice variant of interest identified using the materials and methods of the present invention. Such methods or assays may utilize any means of monitoring or detecting the desired activity.

In one format, the relative amounts of a protein translated from a splice variant of the invention produced in a cell population that has been exposed to the agent to be tested may be compared to the amount produced in an un-exposed control cell population. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, such as a specific antibody.

Probe Design

Probes based on the sequences of splice variants to be detected may be prepared by any commonly available method. Oligonucleotide probes for assaying a tissue or cell sample are preferably of sufficient length to specifically hybridize only to appropriate, complementary transcripts. Typically the oligonucleotide probes will be at least about 10, 12, 14, 16, 18, 20 or 25 nucleotides in length. In some cases longer probes of at least about 30, 40, 50, 60, 70, 80, 90 or 100 or more nucleotides will be desirable.

One of skill in the art will appreciate that an enormous number of array designs are suitable for the practice of this invention. The high-density array will typically include a number of probes that specifically hybridize to one or more splice junctions of interest. In some embodiments, the arrays may further comprise other sequences specific for various parts of the gene of interest, for example, intron or exon specific sequences. See WO 99/32660 for methods of producing probes for a given gene or genes. In addition, in a preferred embodiment, the array will include one or more control probes.

High-density array chips of the invention include “test probes.” Test probes may be oligonucleotides that range from about 5 to about 500, preferably about 10 to about 100 nucleotides, more preferably from about 40 to about 80 nucleotides and most preferably from about 50 to about 70 nucleotides in length. In other particularly preferred embodiments, the probes are about 60 nucleotides in length. In another preferred embodiment, test probes are double or single strand DNA sequences. DNA sequences may be isolated or cloned from natural sources or amplified from natural sources using natural nucleic acid as templates. These probes have sequences complementary to particular subsequences of the splice variant that they are designed to detect. Thus, the test probes are capable of specifically hybridizing to the target nucleic acid they are to detect.

In addition to test probes that bind the target nucleic acid(s) of interest, the high-density array can contain a number of control probes. The control probes fall into three categories referred to herein as (1) normalization controls; (2) expression level controls; and (3) mismatch controls.

Normalization controls are oligonucleotide or other nucleic acid probes that are complementary to labeled reference oligonucleotides or other nucleic acid sequences that are added to the nucleic acid sample. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typical expression level control. probes have sequences complementary to subsequences of constitutively expressed “housekeeping genes” including, but not limited to the β-actin gene, the transferrin receptor gene, the GAPDH gene, and the like.

Mismatch controls may also be provided for the probes to the target splice variants, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a twenty-mer, a corresponding mismatch probe may have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. Mismatch probes also indicate whether hybridization is specific or not. For example, if the target is present the perfect match probes should be consistently brighter than the mismatch probes. In addition, if all central mismatches are present, the mismatch probes can be used to detect a mutation. The difference in intensity between the perfect match and the mismatch probe (I_{(PM)}-I_{(MM)}) provides a good measure of the concentration of the hybridized material.

Nucleic Acid Samples

As is apparent to one of ordinary skill in the art, nucleic acid samples used in the methods and assays of the invention may be prepared by any available method or process. Methods of isolating total mRNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I Theory and Nucleic Acid Preparation, Tijssen, (1993) (editor) Elsevier Press. Such samples include RNA samples, but also include cDNA synthesized from a mRNA sample isolated from a cell or tissue of interest. Such samples also include DNA amplified from the cDNA, and RNA transcribed from the amplified DNA. One of skill in the art would appreciate that it may be desirable to inhibit or destroy RNase present in homogenates before homogenates can be used.

Biological samples may be of any biological tissue or fluid or cells from any organism as well as cells raised in vitro, such as cell lines and tissue culture cells. Frequently the sample will be a “clinical sample” which is a sample derived from a patient. Typical clinical samples include, but are not limited to, sputum, blood, blood cells (e.g., white cells), tissue or fine needle biopsy samples, urine, peritoneal fluid, and pleural fluid, or cells therefrom.

Biological samples may also include sections of tissues, such as frozen sections or formalin fixed sections taken for histological purposes.

Solid Supports

Solid supports containing oligonucleotide probes for use in the present invention can be any solid or semisolid support material known to those skilled in the art. Suitable examples include, but are not limited to, membranes, filters, tissue culture dishes, polyvinyl chloride dishes, beads, test strips, silicon or glass based chips and the like. Suitable glass wafers and hybridization methods are widely available, for example, those disclosed by Beattie (WO 95/11755). Any solid surface to which oligonucleotides can be bound, either directly or indirectly, either covalently or non-covalently, can be used. In some embodiments, it may be desirable to attach some oligonucleotides covalently and others non-covalently to the same solid support.

A preferred solid support is a high-density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 of such features on a single solid support. The solid support or the area within which the probes are attached may be on the order of a square centimeter.

Oligonucleotide probe arrays for expression monitoring can be made and used according to any technique known in the art (see for example, Lockhart et al., Nat. Biotechnol. (1996) 14, 1675-1680; McGall et al., Proc. Nat. Acad. Sci. USA (1996) 93, 13555-13460). Such probe arrays may contain at least two or more oligonucleotides that are complementary to or hybridize to all or a portion of a predicted splice junction. Such arrays my also contain oligonucleotides that are complementary or hybridize to at least 3, 4, 5, 6, 7, 8, 9, 10, 20, 30, 50, 70 or more predicted splice junction sequences.

Oligonucleotide arrays are particularly useful for creating splice variant expression profiles comparing disease tissue to adjacent normal tissue.

The use of oligonucleotide arrays of the invention will enable the determination of the expression levels of numerous splice variants simultaneously. From this mass of expression data, differentially expressed splice variants may be identified using Fold Change and Gene Signature Differential analysis.

Gene Signature Differential analysis is a method designed to detect mRNAs—i. e., splice variants—present in one sample set, and absent in another. mRNAs with differential expression in disease tissue versus normal tissue may be better diagnostic and therapeutic targets than those that do not change in expression.

Methods of forming high-density arrays of oligonucleotides with a minimal number of synthetic steps are known. The oligonucleotide analogue array can be synthesized on a solid substrate by a variety of methods, including, but not limited to, light-directed chemical coupling, and mechanically directed coupling (see Pirrung et al., (1992) U.S. Pat. No. 5,143,854; Fodor et al., (1998) U.S. Pat. No. 5,800,992; Chee et al., (1998) U.S. Pat. No. 5,837,832

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups that are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences has been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in Fodor et al., (1993). WO 93/09668. High-density nucleic acid arrays can also be fabricated by depositing premade or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Hybridization

Nucleic acid hybridization simply involves contacting a probe and target nucleic acid under conditions where the probe and its complementary target can form stable hybrid duplexes through complementary base pairing (see Lockhart et al., (1999) WO 99/32660). The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA-DNA, RNA-RNA or RNA-DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions may be selected to provide any degree of stringency. In a preferred embodiment, hybridization is performed at low stringency, in this case in 6×SSPE-T at 37° C. (0.005% Triton x-100) to ensure hybridization and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPET at 37° C. to 50° C. until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Signal Detection

The hybridized nucleic acids are typically detected by detecting one or more labels attached to the sample nucleic acids. The labels may be incorporated by any of a number of means well known to those of skill in the art (see Lockhart et al., (1999) WO 99/32660).

Databases

The present invention includes relational databases containing sequence information, for instance for one or more splice variants, as well as expression level information in various normal and/or disease tissue samples. Databases may also contain information associated with a given sequence or tissue sample such as descriptive information about the gene associated with the sequence information, or descriptive information concerning the clinical status of the tissue sample, or the patient from which the sample was derived. The database may be designed to include different parts, for instance a sequences database and an expression level database. Methods for the configuration and construction of such databases are widely available, for instance, see Akerblom et al., (1999) U.S. Pat. No. 5,953,727, which is specifically incorporated herein by reference in its entirety.

The databases of the invention may be linked to an outside or external database. In a preferred embodiment, the external database is GenBank and the associated databases maintained by the National Center for Biotechnology Information (NCBI).

Any appropriate computer platform may be used to perform the necessary comparisons between sequence information, expression level information and any other information in the database or provided as an input For example, a large number of computer workstations are available from a variety of manufacturers, such as those available from Silicon Graphics. Client-server environments, database servers. and networks are also widely available and appropriate platforms for the databases of the invention.

The databases of the invention may be used to produce, among other things, electronic Northerns to allow the user to determine the cell type or tissue in which one or more given splice variants are expressed and to allow determination of the abundance or expression level of one or more given splice variants in a particular tissue or cell.

The databases of the invention may also be used to present information identifying the expression level in a tissue or cell of a set of splice variants for a gene, i. e., a transcriptome. Such presentation may comprise comparing the expression level of at least one splice variant in the tissue to the level of expression of the splice variant in the database. Such methods may be used to predict the physiological state of a given tissue by comparing the level of expression of one or more splice variants from one or more genes from a sample to the expression levels found in normal tissue and/or disease tissue. Such methods may also be used in the drug or agent screening assays as described herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

Tissue Sample Acquisition and Analysis

For tissue specimens, samples from normal and/or disease tissue may be used. The samples may be treated using standard techniques. Briefly, frozen tissue may be ground to powder, total RNA extracted using Trizol (Life Technologies), and mRNA isolated using the Oligotex mRNA Midi kit (Qiagen). If necessary, the mRNA may be concentrated using an ethanol precipitation step. Double stranded cDNA may be created using the SuperScript Choice system (Gibco-BRL). cRNA may be synthesized according to standard procedures. To biotin label the cRNA, nucleotides Bio-11-CTP and Bio-16-UTP (Enzo Diagnostics) may be added to the reaction. The cRNA may then be fragmented (5× fragmentation buffer: 200 mM Tris-Acetate (pH 8.1), 500 mM KOAc, 150 mM MgOAc) for thirty-five minutes at 94° C.

Fragmented cRNA may be hybridized to the DNA chips of the present invention under suitable conditions. Such conditions include twenty-four hours at 60 rpm in a 45° C. hybridization oven. The chips may be washed and stained with Streptavidin Phycoerythrin (SAPE) (Molecular Probes) in fluidics stations. To amplify staining, SAPE solution may be added twice with an anti-streptavidin biotinylated antibody (Vector Laboratories) staining step in between. Hybridization to the probe arrays may be detected by fluorometric scanning (Hewlett Packard Gene Array Scanner). Following hybridization and scanning, the microarray images may be analyzed for quality control, looking for major chip defects or abnormalities in hybridization signal. After all chips pass QC, the data may be analyzed using ant available software or data mining tools.

Each DNA chip of the present invention may contain a plurality of oligonucleotide probe pairs per sequence to be detected, for example, splice junction, exon and in some instances, intron sequence. These probe pairs may include perfectly matched sets and mismatched sets, both of which are necessary for the calculation of the average difference. The average difference is a measure of the intensity difference for each probe pair, calculated by subtracting the intensity of the mismatch from the intensity of the perfect match. This takes into consideration variability in hybridization among probe pairs and other hybridization artifacts that could affect the fluorescence intensities. The presence or absence of the various sequences will be used to determine the presence or absence of particular splice variants in a particular sample.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

What is claimed is:

1. A solid support comprising a plurality of oligonucleotides, wherein the plurality comprises oligonucleotides having a sequence that specifically hybridize to a splice junction sequence in a mRNA transcribed from at least one gene, wherein the plurality comprises for each gene at least
$${\sum }_{x = 1}^{n - 1} ( n - x ) + n$$
oligonucleotides, wherein n=the number of exons in each gene.

2. A solid support according to claim 1, further comprising an oligonucleotide that specifically hybridizes to an exon of said gene.

3. A solid support according to claim 1, further comprising an oligonucleotide that specifically hybridizes to an intron of said gene.

4. A solid support comprising at least two oligonucleotides, wherein a first oligonucleotide specifically hybridizes to a splice junction in a first mRNA transcribed from a first gene of interest comprising introns and exons, and a second oligonucleotide specifically hybridizes to a splice junction in a second mRNA transcribed from a second gene of interest comprising introns and exons.

5. A solid support according to claim 4, wherein the first and the second mRNAs are transcribed from different genes.

6. A solid support according to claim 4, wherein the first mRNA and the second mRNA have at least one exon in common.

7. A solid support according to claim 5, further comprising a third and a fourth oligonucleotide, wherein the third oligonucleotide specifically hybridizes to an intron or an exon of the first gene and the fourth oligonucleotide specifically hybridizes to an intron or an exon of the second gene.

8. A solid support comprising oligonucleotides, wherein the oligonucleotides comprise at least one oligonucleotide that specifically hybridizes to each possible splice junction in a mRNA transcribed from a first gene of interest.

9. A solid support according to claim 8, further comprising additional oligonucleotides, wherein the additional oligonucleotides comprise at least one oligonucleotide that specifically hybridizes to each possible splice junction in an mRNA transcribed from a second gene of interest.

10. A method of detecting alternative spliced mRNA, comprising:
contacting a solid support according to any one of claims 1, 4, or 8 with a solution comprising nucleic acids representative of mRNA in a cell; and
detecting an alternatively spliced mRNA.

11. A method according to claim 10, wherein the nucleic acids are ribonucleic acids.

12. A method according to claim 10, wherein the nucleic acids are deoxyribonucleic acids.

13. A method of detecting a pathological condition in a patient, wherein the pathological condition is characterized by alternative splice variants of one or more genes, comprising:
contacting a sample from the patient with a solid support according to any one of claims 1, 4, or 8; and
detecting a level of expression of an alternative splice variant in the sample, wherein the expression level of the alternative splice variant is indicative of a pathological condition.

14. A computer system, comprising:
a database containing information identifying an expression level for one or more alternative splice variants of one or more mRNAs; and
a user interface to view the information.

15. A computer system according to claim 14, wherein the database further comprises information identifying an expression level for an alternative splice variant in normal tissue.

16. A method of identifying an agent that modulates a pathological condition, comprising:
contacting a sample with the agent; and
determining a splice variant profile for at least one gene;
comparing the splice variant profile to a splice variant profile obtained from a sample not treated with the agent; and
determining a change in the splice variant profile, wherein a change in the splice variant profile is indicative of an agent that modulates the condition.

17. An agent identified by the method of claim 16.

18. A pharmaceutical composition comprising an agent according to claim 17 and a pharmaceutically acceptable diluent.

19. A set of oligonucleotides comprising at least one oligonucleotide that specifically hybridizes to each possible splice junction in a mRNA transcribed from at least one gene of interest.

20. A set of oligonucleotides of claim 19, comprising at least
$${\sum }_{x = 1}^{n - 1} ( n - x ) + n$$
oligonucleotides, wherein n=the number of exons in each gene.

21. A set of oligonucleotides of claim 19, comprising at least
$$2 [ {\sum }_{x = 1}^{n - 1} ( n - x ) ] + n$$
oligonucleotides, wherein n=the number of exons in each gene.

22. A set of oligonucleotides of claim 19, comprising oligonucleotides to detect all possible exon-exon junctions between a least two genes.

23. A set of oligonucleotides of claim 22, wherein the set comprises:
$$2 [ {\sum }_{X = 1}^{N - 1} ( N - X ) ] + N + 2 [ {\sum }_{X = 1}^{P - 1} ( P - X ) ] + P + [ N \cdot 2 ( P ) ]$$
oligonucleotides, wherein N=number of exons in a first gene and wherein P=number of exons in a second gene.
